(19) United States
(12) Patent Application Publication
(10) Pub. No.: US 2014/0200175 A1
(43) Pub. Date: Jul. 17, 2014

(54) SUPPRESSION OF TOXIN PRODUCTION OF CLOSTRIDIUM DIFFICLE

(71) Applicants: William T. Self, Orlando, FL (US); Sarah Rosario, Orlando, FL (US)

(72) Inventors: William T. Self, Orlando, FL (US); Sarah Rosario, Orlando, FL (US)

(73) Assignee: University of Central Florida Research Foundation, Inc., Orlando, FL (US)

(21) Appl. No.: 14/022,770

(22) Filed: Sep. 10, 2013

Related U.S. Application Data
(60) Provisional application No. 61/698,950, filed on Sep. 10, 2012.

(51) Int. Cl. C07D 207/16; A61K 38/14; A61K 31/4985; A61K 31/401; C07D 487/04

(52) U.S. Cl. CPC C07D 207/16; A61K 31/401; C07D 487/04; A61K 31/4985; A61K 38/14
USPC 514/3.1; 514/423; 514/249

(57) ABSTRACT

Disclosed in an embodiment herein is a method of treating symptoms of Clostridium difficile in a subject in need, including administering an effective amount of hydroxyproline, or derivative thereof, to the subject, wherein said hydroxyproline, or derivative thereof, inhibits growth or proliferation of Clostridium difficile, or inhibits production of toxins or neutralizes toxins of the bacterium Clostridium difficile.

### CROSS-REFERENCE TO RELATED APPLICATIONS

This application claims priority to U.S. Ser. No. 61/698,950 filed Sep. 10, 2012 which is incorporated herein in its entirety.

### FIELD OF INVENTION

The present invention relates to the field of medical microbiology and, more particularly, to a method of inhibiting growth or proliferation of Clostridium difficile, or inhibiting producing or neutralizing toxins of the bacterium Clostridium difficile. Clostridium difficile is a causative agent of a difficult to treat, nosocomially acquired diarrhea which can progress to pseudo membranous colitis and even death in severe cases.

### BACKGROUND

Clostridium difficile (C. difficile) associated disease is a major health-care related illness with substantial medical and economic consequences and has recently garnered much attention as the causative agent of antibiotic induced diarrhea, leading to severe colitis and even death. Despite the clinical significance of this organism, little is understood regarding the basic metabolic pathways of this organism and their relevance to disease ecology. An outbreak of a more virulent strain (NAP1/O27) in Quebec, Canada in 2001 and the spread of this strain throughout North America and Europe, together with an increasing trend of community acquired illness has firmly established this organism as a significant public health threat (1-6). In the United States, the incidence has more than doubled since the year 2000 and it is estimated that 15,000 to 20,000 people die each year from C. difficile associated disease (7). C. difficile associated disease causes an estimated $1 billion in excess health care costs annually.

In recent years, C. difficile infections have become more frequent, more severe and more difficult to treat. Illness from C. difficile most commonly affects older adults in hospitals or in long term care facilities and typically occurs after use of antibiotic medications. However, each year, tens of thousands of people in the United States get sick from C. difficile, including some otherwise healthy people who aren't hospitalized or taking antibiotics. Recently, the emergence of an epidemic strain that exhibits increased virulence and rising mortality rates in the United States has been of particular concern (McDonald et al., Trifluoroacetic acid as a peptide solvent, J. Biol. Chem. 255: 11199-11203; 2005). C. difficile now rivals methicillin resistant Staphylococcus aureus (MRSA) as a significant clinical pathogen (5, 7, 53). C. difficile is a Gram positive, anaerobic, spore-forming, rod shaped bacterium (a bacillus form) that has emerged a significant nosocomial pathogen. Pathogenesis is mediated by two large clostridial cytotoxins, toxins A and B. C. difficile associated disease generally occurs when antimicrobial therapy disrupts the natural flora of the gastrointestinal tract. The organism colonizes the newly available ecological niche and begins to produce two large molecular weight toxins, toxin A and toxin B. These toxins inactivate GTPases of the Rho/Rac family through glucosyltransferase activity ultimately resulting in depolymerization of the actin cytoskeleton, mucosal damage and inflammation (8). Common symptoms of C. difficile typically range from mild to severe diarrhea, including watery diarrhea three or more times a day for two or more days, and mild abdominal cramping and tenderness. In more severe cases, C. difficile causes the colon to become inflamed or to form patches of raw tissue that can bleed or produce pus (pseudomembranous colitis), signs and symptoms of severe infection include watery diarrhea 10-15 times a day, abdominal cramping and pain which is sometimes severe, fever, blood or pus in stool, nausea, dehydration, loss of appetite, and weight loss. Further complications of C. difficile include toxic megacolon and death (9).

The transcriptional regulation of genes encoding toxins is not well understood, largely due to the recalcitrant nature of genetic studies in C. difficile. Production of toxin varies between strains and based on environmental conditions (10-12). In addition, an increase in the level of toxin produced by the epidemic NAP1/O27 strain has been suggested to play a role in the higher mortality rates observed with this strain (13-15). The toxin genes (tcdA and tcdB) are encoded within the pathogenicity locus (PaLoc) along with two other genes that are involved in regulation of toxin production. tcdC encodes a negative regulator of toxin production that is highly transcribed during exponential growth, but down regulated during stationary phase, the peak of toxin production (16, 17). Polymorphisms in tcdC are associated with increased toxin production (18). tcdR encodes a sigma factor that is positively associated with toxin production (19-22). Another protein encoded outside of the PaLoc, CodY, is implicated in environmental regulation of toxin production (23). C. difficile has a highly variable genome with the core components consisting of only 16% of the encoded genes (24, 25). This creates difficulties in defining the mechanisms of toxin gene regulation and pathogenesis. Additionally, studies of toxin regulation are limited by the resistance of C. difficile to genetic manipulation.

A diverse selection of factors influences toxin production including environmental stress, temperature, biotin limitation and purine biosynthesis (10, 26-28). Of particular interest is the impact of the amino acid composition of the growth medium. Previous studies have shown that regulation of toxin production in C. difficile is altered upon addition or omission of certain amino acids to culture media (11, 12, 29-31). The recent analysis of this regulation showed the addition of L-proline or L-cysteine to the culture medium significantly reduced intracellular toxin levels in a concentration-dependent manner (32), yet the focus was not on extracellular toxin. The molecular mechanism behind this linkage to amino acid metabolism is thus largely unexplained. Moreover, the recurrence rate for C. difficile is approximately 15-35% chance of a relapse for patients with a first episode, within 2 months. (see at: http://www.ncbi.nlm.nih.gov/pmc/articles/PMC86814/). Physiopathy of recurrences may be explained by either the endogenous persistence of C. difficile spores or by the acquisition of a new strain from an exogenous source.

### BRIEF DESCRIPTION

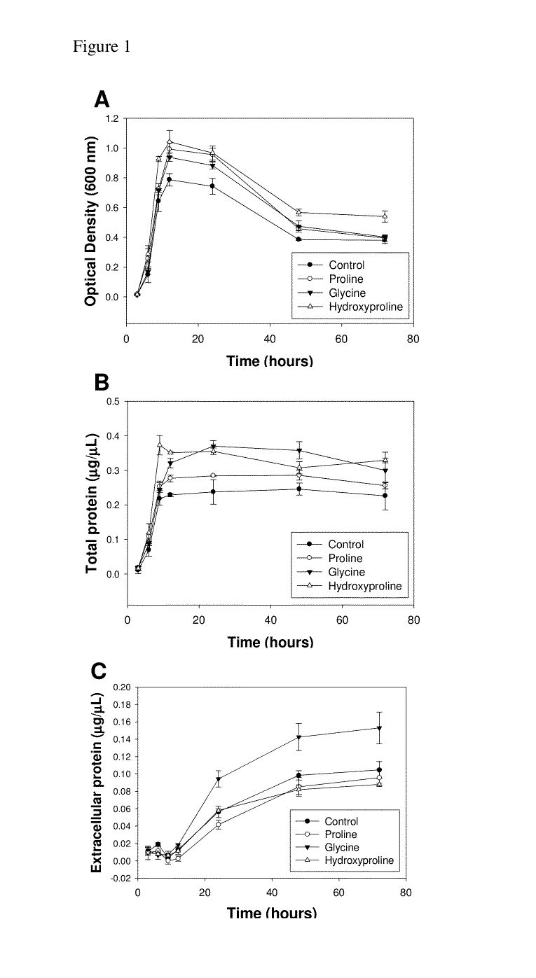

FIGS. 1A-C provides a graphical illustration of the effect of glycine in the production of extracellular protein by C. difficile.

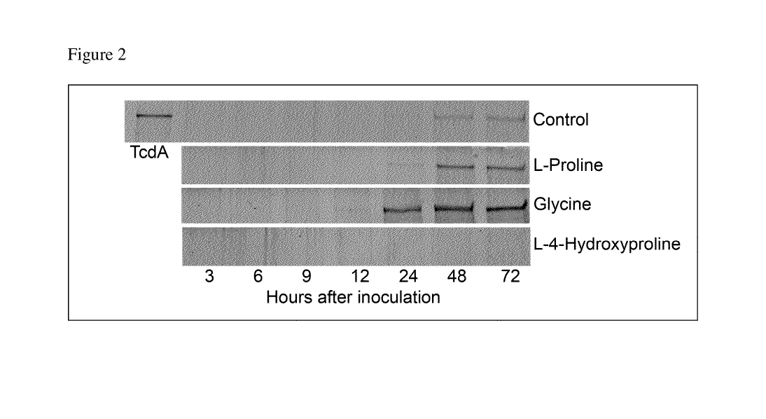

FIG. 2 provides an illustration of the effects of L-proline, glycine, and L-hydroxyproline on the toxin production of C. difficile.

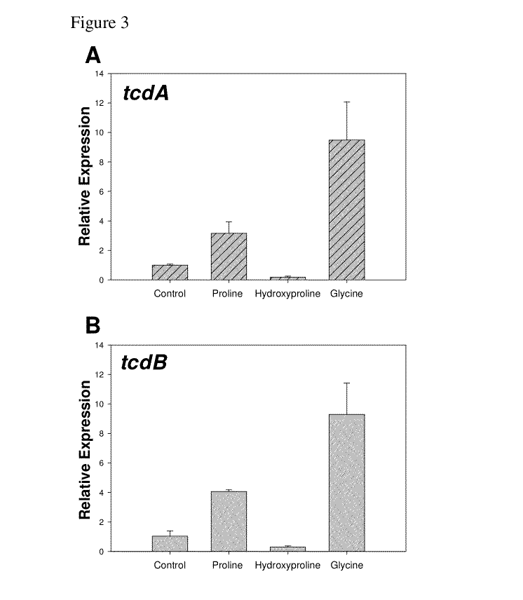

FIGS. 3 A-B provides a graphical illustration of the relationship between the expression of C. difficile toxin mRNA and extracellular toxin production.

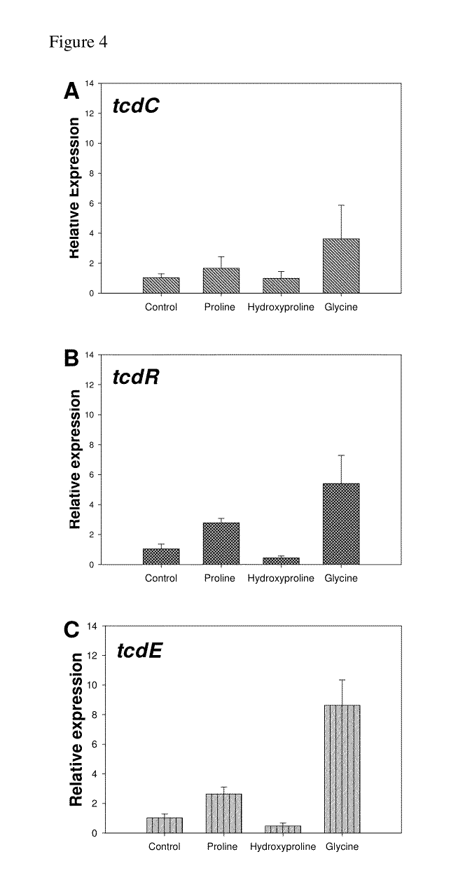

FIGS. 4 A-C provides a graphical illustration of the expression of tcdC, tcdR, and tcdE within the PaLoc following addition of Stickland acceptor amino acids.

### DETAILED DESCRIPTION

Previous work by the inventors described Stickland reactions in C. difficile (33). Stickland reactions are pairwise fermentation of two amino acids in which one is oxidatively deaminated or decarboxylated (Stickland donor) and another amino acid (Stickland acceptor) is reductively deaminated or reduced (34). It should be noted that other sources of reducing potential, such as NADH from oxidation of sugars, can also feed electrons into the selenium-dependent reductases (35). This coupled amino acid fermentation has been demonstrated as a primary source of ATP generation in several organisms including C. sporogenes, C. sticklandii and E. acidaminophilum (36-41). The enzymes responsible for amino acid reduction in C. difficile are the selenoenzymes D-proline reductase (D-PR) and glycine reductase (GR) (33). D-PR catalyzes the reduction of D-proline to 6-aminovaleric acid which is secreted into the growth medium (40, 41). It is suggested that this reaction is considered anaerobic respiration since some studies in the related organism C. botulinum have demonstrated the generation of proton motive force in response to the addition of L-proline (42).

GR, using reducing potential from thioredoxin, deaminates glycine and activates it to acetyl phosphate which can be utilized for substrate level phosphorylation or generation of biomass through production of acetyl-CoA (36, 43). Interestingly, both D-PR and GR are highly conserved across strains and are considered to be part of the core genome of C. difficile (24). Recent proteomic analysis indicates that these enzymes are present within the C. difficile spore (44). In addition, glycine has been shown to be a potent spore co-germinant alongside taurocholate (45). It is thus discovered for the first time herein that amino acid fermentation plays a critical role in transmission of the disease. The impact of Stickland fermentation on toxin production in C. difficile is further elucidated below.

It has been found herein that there is a link between Stickland reactions and toxin regulation. This has been examined in the context of known regulators of toxin production in order to provide insight into the regulatory pathways that control pathogenesis in this organism. C. difficile has been shown to ferment amino acids via Stickland reactions, using glycine, L-proline or L-hydroxyproline as terminal electron acceptors via the selenoenzymes glycine reductase and D-proline reductase. The relationship of these specialized metabolic pathways to pathogenesis, as measured by alterations in toxin production was identified herein. Addition of excess glycine or L-proline enhance transcription of the toxin genes (tcdA and tcdB) and stimulate a marked increase in the production of extracellular toxin. This regulation is mirrored in the other genes within the pathogenicity locus (PaLoc). In contrast, addition of L-hydroxyproline decreases transcription of these genes, and subsequently leads to low levels of toxin in the culture medium.

Due to the severity of the symptoms of C. difficile-infected patients, and the high risk of recurrence of the disease as recognized by the inventors, in one embodiment, a method of treating symptoms of Clostridium difficile in a subject in need is provided herein. The method includes administering an effective amount of hydroxyproline, or derivative thereof, to the subject, wherein said hydroxyproline, or derivative thereof, inhibits growth or proliferation of Clostridium difficile, or inhibits production or neutralizes activity of C. difficile toxins. In another embodiment, the symptoms include but are not limited to diarrhea associated with increased numbers of Clostridium difficile vegetative cells in the subject's stools.

In another embodiment, a method of suppressing toxins of Clostridium difficile is provided. The method includes subjecting said Clostridium difficile to hydroxyproline, or derivative thereof. In a further embodiment, the toxins comprise external toxins.

In yet another embodiment, a method of decreasing a recurrence rate of Clostridium difficile in a subject in need is provided wherein upon infection of the subject with Clostridium difficile, an effective amount of hydroxyproline, or derivative thereof, is administered to the subject for two months following infection to decrease the recurrence rate of Clostridium difficile in the subject. While an effective amount of hydroxyproline, or derivative thereof, can be administered to the subject for two months, it may also be administered to the subject for 1-2 months, or 3-6 months depending on the subject, severity of the illness and symptoms, and the likeliness of recurrence as a result of the environment to which the subject is exposed.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

### Derivatives

Further, derivatives of hydroxyproline are also contemplated for use in accordance with the teachings herein. Derivatives may include, but are not limited to, specific substitutions of reactive constituents on or emanating from hydroxyproline (or alternatively, proline), and may include, but are not limited to, one or more of the following: a hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thio, sulfhydryl, thioalkyl, alkylthio, sulfonyl, C₁-C₆ straight or branched chain alkyl, C₂-C₆ straight or branched chain alkenyl or alkynyl, aryl, aralkyl, heteroaryl, carbocycle, or heterocycle group or moiety, or CO₂ R⁷ where R⁷ is hydrogen or C₁-C₉ straight or branched chain alkyl or C₂-C₉ straight or branched chain alkenyl group or moiety.

According to one embodiment, hydroxyproline derivatives are represented by the following general formulas (I), (II) and (III) or their salts.

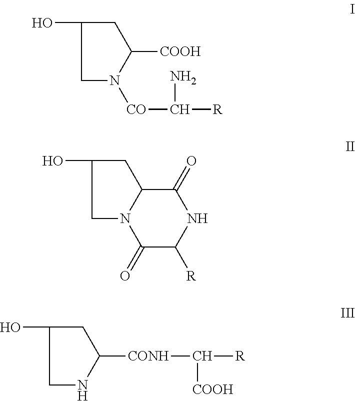

(wherein R is hydrogen or alkyl group which may be substituted with hydroxy group, amino group, carboxy group, aminocarbonyl group, guanidino group, heterocyclic group, mercapto group, alkylthio group or phenyl group optionally substituted with hydroxy group). Derivatives may also pertain to any metabolites of the base compound or to other derivatives.

In a specific embodiment, hydroxyproline derivatives contemplated for use in accordance with the teachings herein include the cis-hydroxyproline derivatives taught in Vergnon et al., J. Comb. Chem. 2004, 6, 91-98. Accordingly, the derivatives taught in the Vergnon et al. are incorporated herein by reference, as well as the techniques of production. For example, the cis-hydroxyproline derivatives taught in Vergnon et al. can be tested for their potential to inhibit C. difficile growth, proliferation, and/or toxin production; and/or neutralize toxin activity.

According to another embodiment, hydroxyproline derivatives useful in accordance with the teachings herein include those provided by the following formula IV:

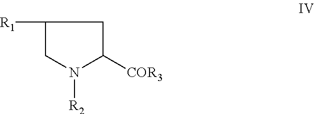

wherein R₁ is a hydrogen, hydroxyl (OH), alkyl, a substituted alkyl group, aryl or amino acid group, amine, or thiol group, R₂ is hydrogen, an alkyl (e.g., C₁-C₄), a substituted alkyl group, a dialkyl, a cyclohexyl, a phenyl or diphenyl group, R₃ is an alkyl group, a substituted alkyl group, a halide, hydrogen, OH, amine, amide; and/or salts thereof. Reference to oxygen in formulas I, II, III, and IV may be substituted by sulfur or nitrogen. Reference to substituted alkyl group includes alkyl group substituted with hydroxy group, amino group, carboxy group, aminocarbonyl group, guanidino group, heterocyclic group, mercapto group, alkylthio group or phenyl group optionally substituted with hydroxy group. In one specific embodiment, when R₁ is a hydroxy group, R₂ is not a methyl group. Methods of producing these compounds is taught, for example, in U.S. Pat. Pub 2008/0176923 and U.S. Pat. No. 7,659,305, whose teachings hydroxyproline derivatives and methods or production are incorporated herein.

In addition, hydroxyproline derivatives are taught in the following U.S. patent documents: U.S. Pat. Nos. 3,891,765; 5,380,875; 6,635,620; 6,497,889; 3,932,638; 3,997,559; 2010/0055062; and 2008/0176923. The teachings of these references are incorporated herein in regard to the hydroxyproline derivatives taught, and the methods of producing such derivatives.

Accordingly, hydroxyproline or derivatives thereof may be tested for their ability to inhibit C. difficile growth, proliferation and/or toxin production. Those found to be active are used to treat C. difficile infection and/or inhibit C. difficile toxin, by administering a therapeutically effect amount of a hydroxyproline derivative taught herein.

The compounds taught herein can be provided in the form of salts. For example, such acid salts include, but are not limited to, the following: acetate, adipate, alginate, aspartate, benzoate, benzenesulfonate, bisulfate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate, and in a particularly preferred fashion the salts of said compounds are iodides, bromides and/or chlorides.

### Antibiotic Agents

Reference to antibiotic agents, as used herein, include antibiotics known to kill C. difficile. These include, but are not limited to, metronidazole, rifaximin, fidaxomicin, and vancomycin. In addition, other examples may include antibody antibiotic agents such as those taught in U.S. Patent Pub. 2010/233181; 2010/0233182; and U.S. Pat. Nos. 8,257,709 and 8,236,311. Other examples include antibiotics taught for example in U.S. Pat Pub. 2010/0184649. All of the foregoing references are incorporated herein with respect to teaching of antibiotic agents.

### Dosage

The dose administered to an animal, particularly a human, in accordance with the present invention should be sufficient to effect the desired response in the animal over a reasonable time frame. One skilled in the art will recognize that dosage will depend upon a variety of factors, including the strength of the particular compositions employed, the age, species, condition, and body weight of the animal. The size of the dose also will be determined by the route, timing and frequency of administration as well as the existence, nature, and extent of any adverse side effects that might accompany the administration of a particular composition and the desired physiological effect. It will be appreciated by one of ordinary skill in the art that various conditions or desired results, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached.

The amount of the compound of the invention administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the compound, the route of administration, pharmacological considerations such as the activity, efficacy, pharmacokinetics and toxicology profiles of the compound and any secondary agents being administered, and the like. Patients undergoing such treatment will typically be monitored on a routine basis to determine the effectiveness of therapy.

Continuous monitoring by the physician will insure that the optimal amount of the compound of the invention will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of compound that exhibits the desired effectiveness is administered and, further, that administration is continued only so long as is necessary to successfully achieve the optimum effect.

The respective dose or dose range for administering the pharmaceutical agent of the invention is in an amount sufficient to achieve the desired antimicrobial or toxin inhibiting effect. The dose should not be selected in such a way that undesirable side effects would dominate. In general, the dose will vary with the age, constitution, sex of a patient, and obviously with respect to the severity of a disease. The individual dose can be adjusted both with respect to the primary disease and with respect to ensuing additional complications. The exact dose can be detected by a person skilled in the art, using well-known means and methods, e.g. as a function of the antimicrobial effect, or of the pharmaceutical carriers and the like. Depending on the patient, the dose can be selected individually. For example, a dose of pharmaceutical agent just tolerated by a patient can be one where the local level in plasma or in individual organs ranges from 0.1 to 100,000 μM, preferably between 1 and 1,000 μM. Alternatively, the dose can also be estimated relative to the body weight of the patient. In this event, for example, a typical dose of pharmaceutical agent would be adjusted in a range of more than 0.1 g per kg body weight, preferably between 0.1 and 5,000 g/kg. Furthermore, it is also possible to determine the dose with respect to individual organs rather than the overall patient. For example, this would apply to those cases where the pharmaceutical agent of the invention, incorporated in the respective patient e.g. in a biopolymer, is placed near particular organs by means of surgery. A number of biopolymers capable of liberating the molecules in a desired manner are well-known to those skilled in the art. For example, such a gel may include from 1 to 1000 g of compounds or pharmaceutical agent of the invention per ml gel composition, preferably between 5 and 500 g/ml, and more preferably between 10 and 100 g/ml. In this event, the therapeutic agent will be administered in the form of a solid, gel-like or liquid composition.

### Pharmaceutical Compositions

Various embodiments of the invention are foreseen to have valuable application as constituents of pharmaceutical preparations to treat various conditions generally defined as pathologies. Accordingly, embodiments of the invention also comprise pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol and cellulose acetate. The compositions may be contained in a vial, sponge, syringe, tube, or other suitable container.

As used herein, the term “administering” or “administration” includes but is not limited to oral or intravenous administration by liquid, capsule, tablet, or spray. Administration may be by injection, whether intramuscular, intravenous, intraperitoneal or by any parenteral route. Parenteral administration can be by bolus injection or by continuous infusion. Formulations for injection may be presented in unit dosage form, for example, in ampoules or in multi-dose containers with an added preservative. The compositions may take the form of suspensions, solutions or emulsions in oily or aqueous vehicles and may contain formulatory agents such as suspending, stabilizing and/or dispersing agents. Alternatively the compositions may be in powder form (e.g., lyophilized) for constitution with a suitable vehicle, for example sterile pyrogen-free water, before use. Compositions may be delivered to a female subject by inhalation by any presently known suitable technique including a pressurized aerosol spray, where the dosage unit may be controlled using a valve to deliver a metered amount.

Administration by capsule and cartridges containing powder mix of the composition can be used in an inhaler or insufflator to deliver the particles to the female subject. Still other routes of administration which may be used include buccal, urethral, vaginal, or rectal administration, topical administration in a cream, lotion, salve, emulsion, or other fluid may also be used.

Turning to the Figures, FIGS. 1A-C demonstrates the addition of glycine increases production of extracellular protein by C. difficile. C. difficile (VPI10463) was cultivated in rich medium with the addition of Stickland acceptor amino acids-L-proline, glycine or L-4-hydroxyproline (16.7 mM). Optical density (FIG. 1A) was followed for 72 hours after inoculation. Total protein (FIG. 1B) and extracellular protein (FIG. 1C) were quantified in cell free supernatants according to the method described by Bradford (46).

FIG. 2 illustrates that toxin production occurs earlier and is increased with the addition of L-proline or glycine to the culture media of C. difficile, but is diminished with L-hydroxyproline. C. difficile (VPI10463) culture media was collected by centrifugation at the indicated time points. Cell free supernatants were filtered (0.2 μm filter) and equal volumes (50 μL) were separated by SDS-PAGE and stained overnight with GelCode Blue. Gel migration was compared with that of purified toxin A (List Biologicals).

FIGS. 3 A-B provides a graphical illustration that the expression of C. difficile toxin mRNA is consistent with the observed changes in extracellular toxin production. C. difficile (VPI 10463) mRNA was harvested after 72 hours of growth in rich media supplemented with L-proline, L-4-hydroxyproline or glycine. Expression of tcdA (FIG. 3A) and tcdB (FIG. 3B) was analyzed using real time RT-PCR.

FIG. 4 A-C provides a graphical illustration of expression of the remaining genes within the PaLoc (tcdRBEAC) following addition of the Stickland acceptor amino acids is consistent with co-transcription with tcdA and tcdB. The analysis was performed as in FIG. 3 above.

### EXAMPLES

### Example 1

### Growth with Stickland Acceptors Increases Toxin Production

The impact of additional Stickland acceptors in rich medium on C. difficile growth and protein production was evaluated over a period of seventy-two hours (FIG. 1). The additional amino acids increased growth approximately 20-30% over the control at twelve hours post-inoculation. The largest increase in growth occurred with L-hydroxyproline (FIG. 1A). Similar results were obtained for total protein (FIG. 1B). In contrast, extracellular protein levels within stationary phase (24 to 72 hours) were significantly higher in cultures supplemented with glycine than the control and those supplemented with L-proline or L-hydroxyproline (FIG. 1C)

Media supernatants were analyzed by SDS-PAGE over the course of the 72 hour growth period. Staining with GelCode Blue revealed the presence of a protein band larger than 225 kDa that appeared after the cells entered into stationary phase (FIG. 2). This corresponded with the migration pattern of the purified toxin A control. The limitations of the 7.5% polyacrylamide gel prevented resolution between toxin A and toxin B. Media supernatants from glycine and L-proline treated cells exhibited higher toxin levels than the control. Glycine treated cells also produced toxin approximately 24 hours earlier. Strikingly no band corresponding to toxin was visible in media supernatants from cells treated with L-hydroxyproline despite the fact that these cultures exhibited comparable growth to glycine and L-proline treated cells. This data corresponds with the results obtained from cell culture based cytotoxicity assays and ELISA (data not shown).

### Example 2

### Stickland Acceptors Enhance Transcription of Toxin Genes

Real time RT-PCR was used to determine if the observed changes in toxin levels in the extracellular growth medium are due to transcriptional regulation of the toxin genes, tcdA and tcdB (FIG. 3). mRNA was harvested after 72 hours of growth. Relative to control cells, those grown with additional L-proline exhibited approximately three times as much transcript for tcdA and tcdB. Those treated with glycine produced nearly nine times as much toxin mRNA. Cells grown with L-hydroxyproline exhibited at least a 75% reduction in tcdA and tcdB transcription. These results confirm that the variations in the cytotoxicity of the growth medium and the amounts of toxin visible by SDS-PAGE analysis are due to transcriptional regulation rather than a post-translational effect. Similar results were obtained with the NAP1/O27 epidemic strain (data not shown).

### Example 3

### The Remaining Genes within the PaLoc are Co-Regulated with Toxins in Response to Stickland Acceptors

Two genes within the PaLoc are reported to play important roles in toxin regulation-tcdC, encoding a putative negative regulator, and tcdR, encoding a sigma factor associated with toxin production. We utilized real time RT-PCR to determine the level of mRNA transcribed from these genes, and tcdE, which is also located within the PaLoc, in response to the addition of Stickland acceptors to the growth medium. Transcription of tcdC is known to exhibit an inverse relationship to toxin transcript levels (17). If the observed transcriptional regulation of tcdA and tcdB in response to additional amino acids were mediated by the product of tcdC, it would be expected that tcdC transcription itself would be decreased in cells treated with L-proline and glycine. However to our surprise the changes that occurred in the level of tcdA and tcdB mRNA were mirrored in the tcdC locus as well (FIG. 4A). The patterns of tcdD and tcdE transcription also mirrored that of tcdA and tcdB (FIGS. 4B and C). This parallel regulation may be attributed to the polycistronic nature of tcdRBEAC transcription (17, 47).

The addition of Stickland acceptors to the growth media of C. difficile has a clear impact on toxin production. Exposure to excess amounts of L-proline and glycine increase toxin production, whereas high levels of L-hydroxyproline decreases the amount of toxin released into the media. Most studies of this organism are focused on epidemiology and inter-strain variation. Due to the lack of available genetic tools in studying C. difficile, the current available knowledge of this organism is limited. Recent advances in this area open up many avenues of study (23, 52, 53). The findings herein further demonstrate the importance of Stickland fermentation in the ecology of C. difficile disease. In particular, recent studies have suggested a role for this metabolic pathway in germination, suggesting that amino acid fermentation may provide the energy required for the cell to exit the dormant state (44).

### Materials and Methods for Examples 1-3

Growth of C. difficile

A high toxin producing strain, VPI 10463 (ATCC, Manassas, Va.), as well as a recent clinical isolate, NAP1/O27 (Dr. Michel Warny, Acambis Inc, Cambridge, Mass.) were used in this study. For routine growth and maintenance cultures were grown in Brain Heart Infusion (BHI, Oxoid) with the addition of 0.5 g/L cysteine. For all experiments assessing toxin regulation and production, C. difficile was cultivated in a rich medium supplemented with selenium (2% tryptone, 0.5% yeast extract, 0.3% glucose, 1 μM sodium selenite, hereafter termed TYGSe). After autoclaving, the culture medium was transferred to an anaerobic chamber with an atmosphere containing 95% nitrogen and 5% hydrogen (Coy Laboratories, Grass Lake, Mich.) and Na₂S was added to a final concentration of 0.03% to pre-reduce the medium. Where indicated, growth medium was supplemented with 16.7 mM L-proline, glycine, or L-4-hydroxyproline. A 1% inoculum from an overnight culture grown in the TYGSe medium was used in all experiments. Cultures were incubated in an anaerobic chamber at 37° C. (Model 2002 incubator, Coy Labs, Great Lakes, Mich.).

### Growth Curve and Extracellular Protein Analysis

Optical density measurements of cultures at 600 nm were determined using a Molecular Devices SpectraMax 190 96-well plate reader at the times indicated for each experiment. Cultures were centrifuged at 16,100×g for five minutes. The resulting cell free supernatants were filtered (0.2 μm) and assayed for protein as described by Bradford (46) using bovine serum albumin (Thermo Scientific, Rockford, Ill.) as a standard.

For analysis of extracellular toxins, 50 μL aliquots of cell free supernatant from each C. difficile culture across a 72 hour growth study was analyzed by sodium dodecyl sulfate-polyacrylamide gel electrophoresis (SDS-PAGE) (7.5% polyacrylamide). The separated proteins were visualized after staining overnight with GelCode Blue (Thermo Scientific, Waltham, Mass.).

### Real Time RT-PCR Analysis of Toxin Gene Expression

C. difficile was cultivated as described above with or without the addition of Stickland acceptors (L-proline, L-4-hydroxyproline or glycine) at a final concentration of 16.7 mM. Seventy-two hours following inoculation cells were harvested by centrifugation (5000×g). Cells were subsequently washed with diethylpyrocarbonate (DEPC) treated phosphate buffered saline (PBS). Total RNA was isolated utilizing the ChargeSwitch Total RNA Cell kit (Invitrogen, Carlsbad, Calif.) and quantified by UV-visible spectrophotometry at 260 nm. cDNA was generated using 1 μg of purified RNA utilizing the iScript cDNA synthesis kit (BioRad, Hercules, Calif.). Real-time PCR amplification was performed utilizing the BioRad i-Cycler. Primer pairs specific to tcdA and tcdB have been described previously (18). Amplification of the transcripts for 16S rRNA were used as an internal standard and this primer pair has also been described for C. difficile (19). Primer pairs for tcdC, tcdD, and tcdE were designed with the Primer 3 software (http://frodo.wi.mit.edu/cgi-bin/primer3/primer3_www.cgi) utilizing the gene sequences from strain 630 and are listed in Table 3-1. BioRad iQ SYBR green supermix was utilized for real-time PCR amplification with primers at a concentration of 250 nM each. cDNA was diluted in sterile H₂O before addition to the reaction mix. Reaction conditions consisted of a single cycle at 95.0° C. for 3 min; subsequent 40 cycles of 95.0° C. for 10 s, 55.0° C. for 45 s. Melt curve analysis and agarose gel electrophoresis were performed to confirm the presence of a single product of expected size. Efficiency of amplification for each target gene was calculated utilizing a 10-fold dilution series of control cDNA. Relative expression was calculated according to the method described previously (20).

### ELISA for Toxins A and B

Culture media was collected by centrifugation (1 min, 16,100×g) after 72 hours of growth. Cell free supernatants were filtered to remove any remaining cell debris (0.2 μm filter). Quantification of extracellular toxins was performed utilizing the C. difficile TOX A/B II ELISA (TechLab, Blacksburg, Va.). Samples were diluted in the diluent provided to obtain readings (O.D.₄₅₀.) within the acceptable range specified by the kit.

TABLE 1
Oligonucleotide primers used for Real Time RT-PCR

| Gene of | | |
|---|---|---|
| Interest | Direction | Sequence |
| tcdA^{a} | forward | tctaccactgaagcattac |
| | | SEQ ID NO. 1 |
| | reverse | taggtactgtaggtttattg |
| | | SEQ ID NO. 2 |
| tcdB^{a} | forward | atatcagagactgatgag |
| | | SEQ ID NO. 3 |
| | reverse | tagcatattcagagaatattgt |
| | | SEQ ID NO. 4 |
| tcdC | forward | ccatggttcacgatcagaca |
| | | SEQ ID NO. 5 |
| | reverse | tgaagaccatgaggaggtca |
| | | SEQ ID NO. 6 |
| tcdD | forward | aactcagtagatgatttgcaagaaa |
| | | SEQ ID NO. 7 |
| | reverse | tctgtttctccctcttcataatgt |
| | | SEQ ID NO. 8 |
| tcdE | forward | tgtgcttatgtggattaccagt |
| | | SEQ ID NO. 9 |
| | reverse | ttcatttcatctgtcattgcatc |
| | | SEQ ID NO. 10 |
| 16S^{b} | forward | ttgagcgatttacttcggtaaaga |
| | | SEQ ID NO. 11 |
| | reverse | ccatcctgtactggctcacct |
| | | SEQ ID NO. 12 |

^{a}described previously (4)
^{b}described previously (32)

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

### REFERENCES

(1) Bartlett, J. G., and Perl, T. M. (2005) The new Clostridium difficile—what does it mean? N Engl J Med, 353, 2503-2505.
(2) Eggertson, L. (2005) Quebec's official numbers: 409 C. difficile deaths. Cmaj, 173, 1308.
(3) (2005) Severe Clostridium difficile-associated disease in populations previously at low risk—four states, 2005. MMWR Morb Mortal Wkly Rep, 54, 1201-1205.
(4) Loo, V. G., Poirier, L., Miller, M. A., Oughton, M., Libman, M. D., Michaud, S., Bourgault, A. M., Nguyen, T., Frenette, C., Kelly, M., Vibien, A., Brassard, P., Fenn, S., Dewar, K., Hudson, T. J., Horn, R., Rene, P., Monczak, Y., and Dascal, A. (2005) A predominantly clonal multi-institutional outbreak of Clostridium difficile-associated diarrhea with high morbidity and mortality. N Engl J Med, 353, 2442-2449.
(5) McDonald, L. C., Killgore, G. E., Thompson, A., Owens, R. C., Jr., Kazakova, S. V., Sambol, S. P., Johnson, S., and Gerding, D. N. (2005) An epidemic, toxin gene-variant strain of Clostridium difficile. N Engl J Med, 353, 2433-2441.
(6) Warny, M., Pepin, J., Fang, A., Killgore, G., Thompson, A., Brazier, J., Frost, E., and McDonald, L. C. (2005) Toxin production by an emerging strain of Clostridium difficile associated with outbreaks of severe disease in North America and Europe. Lancet, 366, 1079-1084.
(7) Rupnik, M., Wilcox, M. H., and Gerding, D. N. (2009) Clostridium difficile infection: new developments in epidemiology and pathogenesis. Nat Rev Microbiol, 7, 526-536.
(8) Just, I., and Gerhard, R. (2004) Large clostridial cytotoxins. Rev Physiol Biochem Pharmacol, 152, 23-47.
(9) Kelly, C. P., and LaMont, J. T. (1998) Clostridium difficile infection. Annu Rev Med, 49, 375-390.
(10) Onderdonk, A. B., Lowe, B. R., and Bartlett, J. G. (1979) Effect of environmental stress on Clostridium difficile toxin levels during continuous cultivation. Appl Environ Microbiol, 38, 637-641.
(11) Yamakawa, K., Kamiya, S., Meng, X. Q., Karasawa, T., and Nakamura, S. (1994) Toxin production by Clostridium difficile in a defined medium with limited amino acids. J Med Microbiol, 41, 319-323.
(12) Karasawa, T., Maegawa, T., Nojiri, T., Yamakawa, K., and Nakamura, S. (1997) Effect of arginine on toxin production by Clostridium difficile in defined medium. Microbiol. Immunol, 41, 581-585.
(13) Eggertson, L. (2004) C. difficile: by the numbers. Cmaj, 171, 1331-1332.
(14) Loo, V. G., Libman, M. D., Miller, M. A., Bourgault, A. M., Frenette, C. H., Kelly, M., Michaud, S., Nguyen, T., Poirier, L., Vibien, A., Horn, R., Laflamme, P. J., and Rene, P. (2004) Clostridium difficile: a formidable foe. Cmaj, 171, 47-48.
(15) Valiquette, L., Low, D. E., Pepin, J., and McGeer, A. (2004) Clostridium difficile infection in hospitals: a brewing storm. Cmaj, 171, 27-29.
(16) Matamouros, S., England, P., and Dupuy, B. (2007) Clostridium difficile toxin expression is inhibited by the novel regulator TcdC. Mol Microbiol, 64, 1274-1288.
(17) Hundsberger, T., Braun, V., Weidmann, M., Leukel, P., Sauerborn, M., and von Eichel-Streiber, C. (1997) Transcription analysis of the genes tcdA-E of the pathogenicity locus of Clostridium difficile. Eur J Biochem, 244, 735-742.
(18) Spigaglia, P., and Mastrantonio, P. (2002) Molecular analysis of the pathogenicity locus and polymorphism in the putative negative regulator of toxin production (TcdC) among Clostridium difficile clinical isolates. J Clin Microbiol, 40, 3470-3475.
(19) Moncrief, J. S., Barroso, L. A., and Wilkins, T. D. (1997) Positive regulation of Clostridium difficile toxins. Infect Immun, 65, 1105-1108.
(20) Mani, N., Lyras, D., Barroso, L., Howarth, P., Wilkins, T., Rood, J. I., Sonenshein, A. L., and Dupuy, B. (2002) Environmental response and autoregulation of Clostridium difficile TxeR, a sigma factor for toxin gene expression. J Bacteriol, 184, 5971-5978.
(21) Dupuy, B., and Matamouros, S. (2006) Regulation of toxin and bacteriocin synthesis in Clostridium species by a new subgroup of RNA polymerase sigma-factors. Res Microbiol, 157, 201-205.
(22) Dupuy, B., Raffestin, S., Matamouros, S., Mani, N., Popoff, M. R., and Sonenshein, A. L. (2006) Regulation of toxin and bacteriocin gene expression in Clostridium by interchangeable RNA polymerase sigma factors. Mol Microbiol, 60, 1044-1057.
(23) Dineen, S. S., Villapakkam, A. C., Nordman, J. T., and Sonenshein, A. L. (2007) Repression of Clostridium difficile toxin gene expression by CodY. Mol Microbiol, 66, 206-219.
(24) Janvilisri, T., Scaria, J., Thompson, A. D., Nicholson, A., Limbago, B. M., Arroyo, L. G., Songer, J. G., Grohn, Y. T., and Chang, Y. F. (2009) Microarray identification of Clostridium difficile core components and divergent regions associated with host origin. J Bacteriol, 191, 3881-3891.
(25) Sebaihia, M., Wren, B. W., Mullany, P., Fairweather, N. F., Minton, N., Stabler, R., Thomson, N. R., Roberts, A. P., Cerdeno-Tarraga, A. M., Wang, H., Holden, M. T., Wright, A., Churcher, C., Quail, M. A., Baker, S., Bason, N., Brooks, K., Chillingworth, T., Cronin, A., Davis, P., Dowd, L., Fraser, A., Feltwell, T., Hance, Z., Holroyd, S., Jagels, K., Moule, S., Mungall, K., Price, C., Rabbinowitsch, E., Sharp, S., Simmonds, M., Stevens, K., Unwin, L., Whithead, S., Dupuy, B., Dougan, G., Barrell, B., and Parkhill, J. (2006) The multidrug-resistant human pathogen Clostridium difficile has a highly mobile, mosaic genome. Nat Genet, 38, 779-786.
(26) Karlsson, S., Dupuy, B., Mukherjee, K., Norin, E., Burman, L. G., and Akerlund, T. (2003) Expression of Clostridium difficile toxins A and B and their sigma factor TcdD is controlled by temperature. Infect Immun, 71, 1784-1793.
(27) Yamakawa, K., Karasawa, T., Ohta, T., Hayashi, H., and Nakamura, S. (1998) Inhibition of enhanced toxin production by Clostridium difficile in biotin-limited conditions. J Med Microbiol, 47, 767-771.
(28) Maegawa, T., Karasawa, T., Ohta, T., Wang, X., Kato, H., Hayashi, H., and Nakamura, S. (2002) Linkage between toxin production and purine biosynthesis in Clostridium difficile. J Med Microbiol, 51, 34-41.
(29) Ikeda, D., Karasawa, T., Yamakawa, K., Tanaka, R., Namiki, M., and Nakamura, S. (1998) Effect of isoleucine on toxin production by Clostridium difficile in a defined medium. Zentralbl Bakteriol, 287, 375-386.
(30) Karasawa, T., Ikoma, S., Yamakawa, K., and Nakamura, S. (1995) A defined growth medium for Clostridium difficile. Microbiology, 141 (Pt 2), 371-375.
(31) Karlsson, S., Burman, L. G., and Akerlund, T. (1999) Suppression of toxin production in Clostridium difficile VPI 10463 by amino acids. Microbiology, 145 (Pt 7), 1683-1693.
(32) Karlsson, S., Lindberg, A., Norin, E., Burman, L. G., and Akerlund, T. (2000) Toxins, butyric acid, and other short-chain fatty acids are coordinately expressed and down-regulated by cysteine in Clostridium difficile. Infect Immun, 68, 5881-5888.
(33) Jackson, S., Calos, M., Myers, A., and Self, W. T. (2006) Analysis of proline reduction in the nosocomial pathogen Clostridium difficile. J Bacteriol, 188, 8487-8495.
(34) Stickland, H. S. (1935) The Chemical Reactions by which Cl. sporogenes Obtains its Energy. Biochemical Journal, 28, 1746-1759.
(35) Barker, H. A. (1961) Fermentation of nitrogenous organic compounds, In The Bacteria (Stanier, I. C. G. a. R. Y., Ed.) pp 151-188, Academic Press, New York, N.Y.
(36) Andreesen, J. R., Wagner, M., Sonntag, D., Kohlstock, M., Harms, C., Gursinsky, T., Jager, J., Parther, T., Kabisch, U., Grantzdorffer, A., Pich, A., and Sohling, B. (1999) Various functions of selenols and thiols in anaerobic gram-positive, amino acids-utilizing bacteria. Biofactors, 10, 263-270.
(37) Britz, M. L., and Wilkinson, R. G. (1982) Leucine dissimilation to isovaleric and isocaproic acids by cell suspensions of amino acid fermenting anaerobes: the Stickland reaction revisited. Can J Microbiol, 28, 291-300.
(38) Costilow, R. N. (1977) Selenium requirement for the growth of Clostridium sporogenes with glycine as the oxidant in stickland reaction systems. J Bacteriol, 131, 366-368.
(39) Seto, B., and Stadtman, T. C. (1976) Purification and properties of proline reductase from Clostridium sticklandii. J Biol Chem, 251, 2435-2439.
(40) Stadtman, T. C., and Elliott, P. (1957) Studies on the enzymic reduction of amino acids. II. Purification and properties of D-proline reductase and a proline racemase from Clostridium sticklandii. J Biol Chem, 228, 983-997.
(41) Stadtman, T. C. (1956) Studies on the enzymic reduction of amino acids: a proline reductase of an amino acid-fermenting Clostridium, strain HF. Biochem J, 62, 614-621.
(42) Lovitt, R. W., Kell, D. B., and Morris, J. G. (1986) Proline reduction by Clostridium sporogenes is coupled to vectorial proton ejection, pp 269-273.
(43) Andreesen, J. R. (2004) Glycine reductase mechanism. Curr Opin Chem Biol, 8, 454-461.
(44) Lawley, T. D., Croucher, N.J., Yu, L., Clare, S., Sebaihia, M., Goulding, D., Pickard, D. J., Parkhill, J., Choudhary, J., and Dougan, G. (2009) Proteomic and genomic characterization of highly infectious Clostridium difficile 630 spores. J. Bacteriol.
(45) Sorg, J. A., and Sonenshein, A. L. (2008) Bile salts and glycine as cogerminants for Clostridium difficile spores. J Bacteriol, 190, 2505-2512.
(46) Bradford, M. M. (1976) A rapid and sensitive method for the quantitation of microgram quantities of protein utilizing the principle of protein-dye binding. Anal Biochem, 72, 248-254.
(47) Hammond, G. A., Lyerly, D. M., and Johnson, J. L. (1997) Transcriptional analysis of the toxigenic element of Clostridium difficile. Microb Pathog, 22, 143-154.
(48) Tojo, S., Satomura, T., Kumamoto, K., Hirooka, K., and Fujita, Y. (2008) Molecular mechanisms underlying the positive stringent response of the Bacillus subtilis ilv-leu operon, involved in the biosynthesis of branched-chain amino acids. J Bacteriol, 190, 6134-6147.
(49) Handke, L. D., Shivers, R. P., and Sonenshein, A. L. (2008) Interaction of Bacillus subtilis CodY with GTP. J Bacteriol, 190, 798-806.
(50) Levdikov, V. M., Blagova, E., Joseph, P., Sonenshein, A. L., and Wilkinson, A. J. (2006) The structure of CodY, a GTP— and isoleucine-responsive regulator of stationary phase and virulence in gram-positive bacteria. J Biol Chem, 281, 11366-11373.
(51) Goulding, D., Thompson, H., Emerson, J., Fairweather, N., Dougan, G., and Douce, G. R. (2009) Distinctive profiles of infection and pathology in hamsters infected with C. difficile strains 630 and B1. Infect Immun.
(52) Heap, J. T., Pennington, O. J., Cartman, S. T., Carter, G. P., and Minton, N. P. (2007) The ClosTron: A universal gene knock-out system for the genus Clostridium. J Microbiol Methods.
(53) Lyras, D., O'Connor, J. R., Howarth, P. M., Sambol, S. P., Carter, G. P., Phumoonna, T., Poon, R., Adams, V., Vedantam, G., Johnson, S., Gerding, D. N., and Rood, J. I. (2009) Toxin B is essential for virulence of Clostridium difficile. Nature, 458, 1176-1179.

1. A method of treating symptoms of Clostridium difficile in a subject in need, said method comprising:
administering an effective amount of hydroxyproline or hydroxyproline derivative to the subject, wherein said hydroxyproline or hydroxyproline derivative decreases Clostridium difficile toxin production or suppresses toxin in the subject.

2. The method of claim 1, wherein hydroxyproline or hydroxyproline derivative decreases transcription of toxin genes tcdA and tcdB.

3. The method of claim 1, wherein said symptoms include diarrhea associated with increased numbers of Clostridium difficile vegetative cells in the subject's stools.

4. The method of claim 1, wherein said method suppresses toxins of Clostridium difficile in said patient.

5. The method of claim 4, wherein the toxins comprise external toxins.

6. The method of claim 4, wherein hydroxyproline or hydroxyproline derivative decreases transcription of toxin genes tcdA and tcdB.

7. The method of claim 4, wherein suppressing the toxins of Clostridium difficile prevents the patient from exhibiting a symptom of Clostridium difficile, wherein said symptoms comprise diarrhea, abdominal cramping, pseudomembranous colitis, and/or toxic megacolon.

8. A method of decreasing a recurrence rate of Clostridium difficile in a subject in need, wherein upon infection of the subject with Clostridium difficile, an effective amount of hydroxyproline or hydroxyproline derivative is administered to the subject for at least two weeks following infection to decrease the recurrence rate of Clostridium difficile in the subject.

9. The method of claim 8, wherein hydroxyproline or hydroxyproline derivative is administered to the subject for at least 4 weeks following infection.

10. The method of claim 9, wherein hydroxyproline or hydroxyproline derivative is administered to the subject for at least 8 weeks following infection.

11. The method of claim 8, wherein said infection of the subject with Clostridium difficile caused diarrhea associated with increased numbers of Clostridium difficile vegetative cells in the subject's stools.

12. A method of treating Clostridium difficile infection in a subject; said method comprising
administering a therapeutically effective amount of antibiotic agent to said subject; and
coadministering a therapeutically effective amount of hydroxyproline or hydroxyproline derivative; wherein said hydroxyproline or hydroxyproline derivative, decreases Clostridium difficile toxin production or suppresses toxin in the subject.

13. The method of claim 12, wherein said antibiotic agent is a glycopeptide, such as oritavancin,

14. The method of claim 12, wherein said hydroxyproline or hydroxyproline derivative decreases sporulation of said Clostridium difficile.

15. The method of claim 12, wherein said antibiotic agent and said hydroxyproline or hydroxyproline derivative are provided together in a composition.

16. The method of claim 12, wherein said hydroxyproline derivative is a derivative comprising a specific substitution of a reactive constituent on or emanating from hydroxyproline (or alternatively, proline), and may include, but are not limited to, one or more of the following: a hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thio, sulfhydryl, thioalkyl, alkylthio, sulfonyl, C₁-C₆ straight or branched chain alkyl, C₂-C₆ straight or branched chain alkenyl or alkynyl, aryl, aralkyl, heteroaryl, carbocycle, or heterocycle group or moiety, or CO₂ R⁷ where R⁷ is hydrogen or C₁-C₉ straight or branched chain alkyl or C₂-C₉ straight or branched chain alkenyl group or moiety.

17. (canceled)

18. (canceled)

19. (canceled)

20. (canceled)

21. (canceled)


SEQUENCE LISTING (12 sequences)

SEQ ID NO: 1 - DNA, 19 bp, Artificial sequence
  Synthetic
tctaccactgaagcattac

SEQ ID NO: 2 - DNA, 20 bp, Artificial sequence
  Synthetic
taggtactgtaggtttattg

SEQ ID NO: 3 - DNA, 18 bp, Artificial sequence
  Synthetic
atatcagagactgatgag

SEQ ID NO: 4 - DNA, 22 bp, Artificial sequence
  Synthetic
tagcatattcagagaatattgt

SEQ ID NO: 5 - DNA, 20 bp, Artificial sequence
  Synthetic
ccatggttcacgatcagaca

SEQ ID NO: 6 - DNA, 20 bp, Artificial sequence
  Synthetic
tgaagaccatgaggaggtca

SEQ ID NO: 7 - DNA, 25 bp, Artificial sequence
  Synthetic
aactcagtagatgatttgcaagaaa

SEQ ID NO: 8 - DNA, 24 bp, Artificial sequence
  Synthetic
tctgtttctccctcttcataatgt

SEQ ID NO: 9 - DNA, 22 bp, Artificial sequence
  Synthetic
tgtgcttatgtggattaccagt

SEQ ID NO: 10 - DNA, 23 bp, Artificial sequence
  Synthetic
ttcatttcatctgtcattgcatc

SEQ ID NO: 11 - DNA, 24 bp, Artificial sequence
  Synthetic
ttgagcgatttacttcggtaaaga

SEQ ID NO: 12 - DNA, 21 bp, Artificial sequence
  Synthetic
ccatcctgtactggctcacct